(19) United States
(12) Patent Application Publication
(10) Pub. No.: US 2009/0005837 A1
(43) Pub. Date: Jan. 1, 2009

(54) Method and apparatus for stimulating the neurochemistry of the brain resulting in increased overall brain function, cognitive performance, and intelligence quota

(76) Inventor: Ruth Olmstead, Ft. Lauderdale, FL (US)

Correspondence Address: Larry D. Johnson, P.O. Box 470277, Celebration, FL, 34747, US

(21) Appl. No.: 11/824,288

(22) Filed: Jun. 29, 2007

(51) Int. Cl. A61N 5/06

(52) U.S. Cl. 607/88

(57) ABSTRACT

An apparatus and method for stimulating the neurochemistry of the brain including enzymes, nucleosides, nucleotides, neuropeptides, neurotransmitters, amino acids, brain glucose, steroids, and hormones, and more specifically, an apparatus and method for enhancing overall brain function, including cognitive abilities and intelligence quota (IQ). Other treatment interventions the apparatus and method can positively affect include neurological, physiological, and psychological disorders and diseases. The inventive method utilizes a specially designed goggle which contains two round eye pieces to which are attached the light assemblies. These light assemblies are comprised of 1-20 white full spectrum lights which are directed toward both eyes, within a two inch radius of the retina to avoid overstimulation. The goggle has an expandable nose piece with a mechanism to allow the eye pieces to swing outward and adjust over the eye of any individual, in accordance with the specified retinal placement. The inset lights flash or flicker utilizing intermittent “fuzzy” light, and encompass frequencies ranging from 1-1200 Hz (cycles per second), specifically excluding the range between 55-65 Hz to prevent any form of seizure. The light stimulus is accompanied by unmatched, intermittent sound stimulation, called “fuzzy” sound. The sound is audible through the use of any type headphone. The method is in the form of computerized programs, and includes a variety of formulas translated as “sessions” to be downloaded into any existing light/sound device or apparatus. These formulas are in the form of a variety of sessions, lasting no more than 40 minutes each, to address and or treat a variety of symptoms, disorders, and diseases. The sessions control the light goggle apparatus and sound. Neither the “fuzzy” light nor the “fuzzy” sound are synchronized.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to the field of brain stimulation, and more particularly to increases in brain chemistry for enhancing overall brain function, including cognitive abilities and intelligence quota (IQ), utilizing full spectrum white light applied to both eyes or only to one eye, at frequencies ranging from 1-1200 Hz, omitting 55-65 Hz to avoid seizure. Both “fuzzy” light and “fuzzy” sound are an integral part of the method, and are controlled though computerized sessions, which can be downloaded to any existing light/sound device. More specifically, this is an apparatus and method for enhancing overall brain functions, including cognitive abilities and intelligence quota (IQ). Other treatment interventions this apparatus and method can positively affect include neurological, physiological, and psychological disorders and diseases.

### BACKGROUND INFORMATION AND DISCUSSION OF RELATED ART

Brainwave states are derived from natural frequencies occurring within the cortex, but can be altered through the use of light and sound driven devices. When a light emitting goggle or light frame is worn over the eyes, any lights flickering at specific frequencies for a length of time will cause a reaction in brainwave activity. For example, if the light goggles are set to flicker at 13 Hz for a ten to twenty minute period of time, the brainwave activity will slow or become faster and match, or “entrain” to the 13 Hz frequency. This action is typically called “entrainment” or “frequency following response” (FFR). This is a common process utilized by most light/sound devices to guide the user into desired brainwave states.

Light and sound stimulation therapy utilizes a variety of light/sound devices, to alter brainwave activity, regulate circadian rhythms, and treat seasonal depressive disorder, for example. Light/Sound devices have been employed for improvements in relaxation, cognitive development, peak performance, problem solving abilities, and to increase test scores in learning disabled. children.

Previous animal studies have indicated that animals placed in enriched and stimulating environments result in higher learning and increases in brain weight and density. Effects of stimulation in the human brain have been positive. Postmortem human studies revealed that subjects exposed to challenging and stimulating environments demonstrate greater dendritic length and structural changes in the cortex.

Neurological studies demonstrated that photic driven light stimulation to the brain increased cerebral blood flow and glucose uptake at higher levels than oxygen consumption. Other researchers found statistically significant increase in lactate production during light stimulation, and found that light stimulation caused an increase in brain activity resulting in selectively stimulating glycolysis.

Increasing evidence has demonstrated that changing the cerebral electrical activity associated with learning disabilities improves symptoms and enhances cognitive performance. Light driven electroencephalogram (EEG) neurofeedback demonstrated improved regulation of irregular brainwave states that affect learning and attention, and resulted in increased neuroactivation.

Brainwave frequencies are electrical wave patterns generated by the brain. Delta frequencies are associated with sleep, and range approximately between 5-3 Hz. Theta brainwaves are associated with deep relaxation, and range approximately between 4-7 Hz. Alpha brainwaves are associated with being mentally alert, yet relaxed, and range approximately between 8-12 Hz. Beta brainwave frequencies are associated with being alert in our waking state, and range approximately between 13-34 Hz.

It should be noted that the frequency at approximately 35 Hz is considered to be in the gamma range. Gamma brainwaves are important to higher learning, peak performance, and the increasing evolutionary process of the brain. Studies have demonstrated that faster brainwave activity in the gamma range, and up to 70 Hz heightens during, and may be associated with, the formation of ideas and memory, linguistic processing, and other abilities and behavioral functions.

Currently, available light/sound devices or light emitting goggles are not utilizing frequencies beyond 42 Hz. Current light sound devices or software do not utilize “fuzzy” light or “fuzzy” sound. Current methods do not deliberately omit the 55-65 frequency range within any method as a safety function to avoid seizure by “feedback”.

Various prior art systems have been developed for use in stimulating and patterning brain functions. For example, U.S. Pat. No. 4,315,502 related to an apparatus for stimulating and coordinating whole brain wave function. The apparatus includes a combination of a source of pulsating light within a mask that directs the light sources adjacent the left and right eyes of a subject, and an audio headset that applies auditory signals to the ears of the subject.

U.S. Pat. No. 5,709,645 encompasses an independent field photic stimulator capable of isolating and stimulating the left and right visual fields of each eye independently of each other, but does not utilize a varied “fuzzy” light or sound stimulus, for example.

U.S. Pat. No. 5,465,939 describes a method for assessing and the amelioration of brain function after physical and psychological trauma.

U.S. Pat. No. 5,562,719 conjectures that “light” stimulus provided to the non-dominant cerebral hemisphere is a viable treatment for a variety of biopsychiatric disorders, including Seasonal Affective Disorder (SAD).

The foregoing patents reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein.

### SUMMARY OF THE INVENTION

The present invention provides an apparatus and method for enhancing overall brain functions, including cognitive abilities and intelligence quota (IQ). The present apparatus and method which controls it utilize Hz frequencies ranging between 1-1200 within a specially designed goggle to affect the neurochemistry and physiology of the retina, eye, optic nerve, optic tract, brain, the central nervous center, the regulating systems of the body, and more specifically, increase cognitive function and intelligence quota (IQ). The difference between the inventive method of stimulus and other devices and methods is the specific placement of the lights away from the retina, the frequency of light stimulation, which ranges from 1-1200 Hz, the omission of 55-65 Hz, and the “fuzzy” light and “fuzzy” sound which will be described in further detail.

Individuals with photosensitivity to lights (epilepsy) experience seizure due to an abnormally narrow boundary layer between the visual cortex and the medulla oblongata. Excluding the frequency range of 55-65 Hz excludes the chance of seizure, as there is no chance of equalization of frequencies and feedback.

The goggle or apparatus will deliver the stimulus by utilizing the optic system, administering light to pass through the pupil, and stimulates the rods and cones located in the retina. Once in the retina, the white full spectrum light excites the ganglion cells before passing through the optic nerve. Next, the light travels through the optic chiasm to pass on to either side of the cerebral hemisphere, causing increased neuronal activation to occur.

The light is shaped in the form of square waves, and can be programmed at varying duty cycles and frequencies, and flashes in specific patterns. These different light flickering patterns will have variance of at least 10% to 100% of the frequency wavelength. This variance will be referred to as “fuzzy” light, and result in specific and increased stimulus with regard to neuro chemical and brainwave responses. The object of utilizing “fuzzy” light is also to provide a more controllable form of stimulus to avoid any potential neurological “feedback” loop with regard to stimulus in the brain.

The frequency of the flickering will range from 1-1200 Hz. It should noted that few people perceive light as flickering at approximately 75 Hz, which has been called continuous light.

The prior art light and sound devices currently utilize binaural beats, described as the result of tones at different beats which are simultaneously heard by both ears. The inventive method provides the additional use of “fuzzy” sounds, in the form of tones and beats, which will be audible through the use of high quality headphones. Fuzzy sounds are defined as tones or pulses which utilize a duty cycle that is altered in terms of the length of time in which the tone is on and off, and being slightly indistinct. For example, the tone may be on or audible for 50% of the pulse width, on, and then off for 49% of the pulse width, then 48% on, 50% off, etc. The objects of such “fuzzy” tones are to prevent a repeatable auditory “feedback” with regard to stimulus in brain, and thereby increasing stimulus variance. “Fuzzy” sounds may or may not coordinate with the “fuzzy” light stimulus, thereby providing greater stimulus to the brain and promoting dendrite growth.

The inventive method provides a series of software programs designed as formulas for addressing and treating specific symptoms. The method will be downloaded into a variety of compatible existing light/sound devices. Each computer generated program will not exceed 40 minutes in length, to prevent “over stimulation” of the brain, or fatigue of the “stimulus response”. The inventor theorizes that it is of significant importance to “encourage” the neuronal and psychological response, not to “over-drive” these responses.

The method will be comprised of a variety of software programs specific to the results that the user is trying to achieve, and will embody approximately four main program types; Excitatory, Inhibitory, Excitatory Crossover, and Inhibitory Crossover. Dopamine is increased by the Excitatory Programs. Serotonin is increased by the Inhibitory programs. Endorphin production is increased with the use of both of the Crossover Programs, as they promote a balanced production of both dopamine and serotonin, resulting in an increased endorphin level.

The method and programs are preferably contained in the form of an electronic storage file, which can be provided in a variety of ways including the Internet, wireless technology, or other such means of program material or electronic communications.

The method and apparatus will utilize only full spectrum white light stimulus provided to both hemispheres of the brain through the retina and optic nerves. White full spectrum light is more beneficial to those with a variety of psychological and neuronal disorders. White full spectrum light placed at a 2 inch radius from the retina provides a more effective, “even” distribution of full spectrum or “white” light, causing the brain to “search” and sort out various stimuli from the visual cortex. This type of placement additionally results in an increase in the activation of the brain chemistry. White full spectrum light in this aforementioned placement using the periphery, is also more stimulating as it forces the brain “to search”, develop patterns, and does not over stimulate the rods and cones of the eye as easily as single color lights (red, blue etc.), which additionally decreases the chance of seizure.

The present invention thus provides an apparatus and method for stimulating the neurochemistry of the brain and more specifically, for enhancing overall brain function, including cognitive abilities and intelligence quota (IQ). Other treatment interventions the inventive apparatus and method can positively affect include neurological, physiological, and psychological disorders and diseases. The apparatus is a specially designed goggle which contains light assemblies comprised of 1-20 white full spectrum lights which will be directed toward both eyes, within a two inch radius of the retina to avoid overstimulation.

The inventive method includes visual stimulus introducing unmatched flickering utilizing intermittent or “fuzzy” light and fuzzy sound, which will be unmatched.

The preferred apparatus for carrying out the method of the invention includes a light goggles or eye-frames, positioned in front of the patient's eyes, within a 2 inch radius from the retina. The eye goggles are adjustable at the nose-piece, so that the device can be adapted to accommodate the significant varying interpupillary distances from patient to patient.

The types of lights that are used with the apparatus and method may include white full spectrum light, polarized light, or most preferably, white full spectrum light emitting diodes (L.E.D.'s).

The present invention encompasses a method and apparatus for delivering light and sound stimulation for the treatment of a wide variety of disorders and diseases, such as but not limited to psychiatric, medical, and mental disorders. The invention utilizes light at frequencies from 1-1200 Hz to travel through the optical system, the retina, and its neuronal connections, and the optic nerve that enters the hemisphere of the brain, to excite the neurochemistry of the brain.

It is therefore an object of the present invention to provide a new and improved apparatus and method for enhancing overall brain functions, including cognitive abilities and intelligence quota (IQ).

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front elevation view of a light goggle apparatus of this invention; and

FIG. 2 is a top elevation view of the light goggle apparatus of FIG. 1.

### DETAILED DESCRIPTION OF THE INVENTION

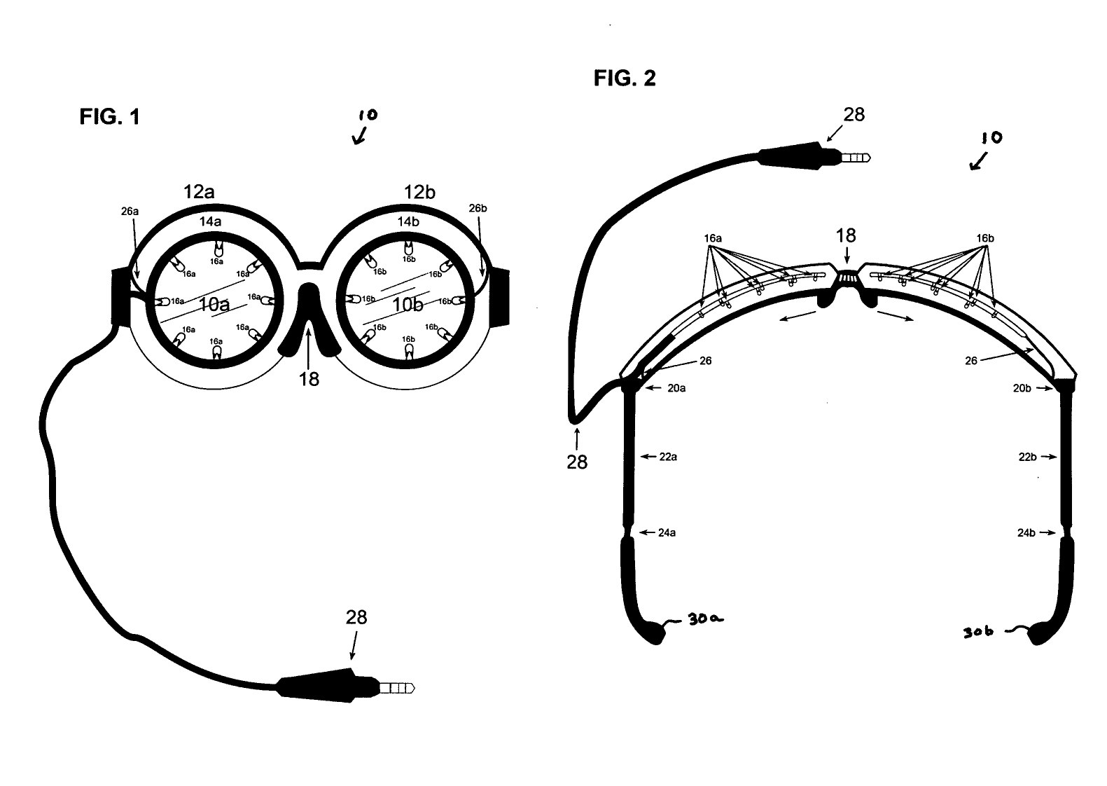

Referring to FIGS. 1 and 2, wherein like reference numerals refer to like components in the various views, there is illustrated therein a new and improved apparatus for enhancing overall brain functions, including cognitive abilities and intelligence quota (IQ), generally denominated 10 herein.

FIG. 1 shows a light emitting goggle 10 comprised of two separate clear, round, and adjustable eye shields 10a, 10b attached to the upper half of the goggle frame 12a, 12b. A second shield 14a, 14b is attached to the inside of each eye shield 10a, 10b and inset with 8 to 20 LED's 16a, 16b, placed in a two inch diameter circular pattern. The secondary shield 14a, 14b has a circular magnet wire 26a, 26b around it, to which the LED's 16a, 16b are attached. The magnet wire attaches to the power cord (28). Although FIG. 1 depicts a goggle with only 8 LED's 16a, 16b in each eye shield, it should be noted that this apparatus can be varied utilizing 8 to 20 LED's, within the two inch diameter circular pattern.

These light emitting goggles 10 will be expandable at the nose piece 18, which will allow the eye shields 10a, 10b to swing outward, in order to more accurately fit the individual face and eyes. The surface of each eye shield 10a, 10b may be, for example, a slightly curved surface. The inside surface of each eye shield 10a, 10b is to be fitted with eight or more white, full spectrum LED's 16a, 16b, or any other source that can carry light such as fiber optics.

FIG. 2 depicts a top elevation view of the goggle apparatus. The goggle frame 12a, 12b will provide hinges 20a, 20b for the temple pieces 22a, 22b which include length adjustment sections 24a, 24b. The LED's 16a, 16b within each eye frame 10a, 10b will be powered with a magnet wire 26 which connects to a three conductor, ⅛ inch mini connector at the opposite end of the cord 28. This cord 28, of various length, will attach to the power output jack on the signal device, not shown, and supplies the voltage and control signals to such a device based on the downloaded software. Earphones 30a, 30b may be incorporated into the temple pieces 22a, 22b to deliver the auditory stimulus to the subject, obviating the need for discrete headphones.

Because color perception is perceived from the periphery of vision, this design specifically places the lights within goggles approximately two inches from the retina (e.g., in the range of 1½ to 2½ inches). This placement is designed to result in superior light optimization, while preventing the over-saturation of the rods and cones of the eye. Additionally, this placement stimulates the eye and brain in a more appropriate manner, as closer stimulus to the iris of the eye results in closure due to the intensity of the light, as well as a constant “over-focusing”, which can result in headache in some individuals. The glasses are expandable across the face with regard to width, and the individual round goggle will swing outward to best fit the width of the face and meet the 2-inch requirement of the light stimulus around the retina. The inside surface is fitted with eight or more white, full spectrum L.E.D.'s, or any other source that can carry light such as fiber optics. FIG. 1 shows 8 lights, mounted evenly in a two inch radius on each eye shield, with two directly to the outside of the eye, and the others evenly spaced around the radius.

Auditory stimulation will be generated by the software, and through the use of any high quality audio headset available on the market. The software programs generate an auditory signal in terms of a tone or pulse up to 22,000 cycles, and with a specific frequency using a digital or analogue method according to the characteristics of the brain wave-inducing signal. The auditory signal is outputted to the earphone through the square wave generating unit, the volume controller and the audio signal-amplifying unit. In this case, the pitch is determined according to characteristics of the auditory signal generated by the program.

One preferred embodiment of the present invention encompasses an apparatus and method for increasing brain chemistry, including enzymes, nucleosides, nucleotides, neuropeptides, neurotransmitters, amino acids, brain glucose, steroids, and hormones.

Another preferred embodiment of the present invention encompasses an apparatus and method to increase cognitive function and overall Intelligence Quota (IQ).

Another preferred embodiment of the present invention encompasses an apparatus and method for positive treatment outcomes of all psychological disorders as listed in the Diagnostic and Statistical Manual of Mental Disorders Revised Fifth Edition, Revised (DSM-V-R).

Another preferred embodiment of the present invention encompasses an apparatus and method for affecting all neurological and dementing diseases and disorders including Alzheimer's and Parkinson's disease.

Another preferred embodiment of the present invention encompasses an apparatus and method for affecting migraine.

Another preferred embodiment of the present invention encompasses an apparatus and method for such, as but not limited to decreasing medication intake resulting from the productions of specific brain chemistry.

Another preferred embodiment of the present invention encompasses an apparatus and method for affecting brain trauma, stroke, and revitalizing comatose patients.

Another preferred embodiment of the present invention encompasses an apparatus and method for increasing neuronal activation of the brain which can “overwrite” and interrupt the “fight-flight” response, resulting the restored psychological and neurological functioning of emotionally traumatized psychiatric patients.

Another preferred embodiment of the present invention encompasses an apparatus and method for the treatment of alcohol and substance abuse. Neurochemical changes can be affected, as well as the psychological component of addiction which may be assisted in the “re-wiring” and interruption of the stimulus-response mechanism.

Another preferred embodiment of the present invention encompasses an apparatus and method for increased hand-eye coordination, which can specifically address and enhance sports performance.

Another preferred embodiment of the present invention encompasses an apparatus and method for pain management. One function of endorphin appears to be mediation of pain perception. When the body experiences pain it uses it own endorphin, which are one of three neuropeptides, which bind to opioid receptors in the brain and have potent analgesic affect. Long term pain can result in a lower level of endorphin production, thus other approaches to manage pain may be greatly beneficial and are greatly needed in the art, the inventor theorizes that the use of the apparatus and method may induce a greater production of endorphin levels, and a significantly decrease pain levels.

Another preferred embodiment of the present invention encompasses an apparatus and method for the increased production of the fat-soluble compound cholecalciferol (q.v.), which is considered a hormone and is synthesized in the skin. The increase and/or synthesis of cholecalciferol with or without ergocalciferol (q.v.) can have profound effects on decreasing cancer cell production and in the reduction of cancerous and non cancerous tumors.

Another preferred embodiment of the present invention encompasses an apparatus and method for medical disorders and diseases of the central nervous system.

Another preferred embodiment of the present invention encompasses an apparatus for providing specially designed light goggles which can be used with or without the programs, and with other light/sound devices.

Another preferred embodiment of the present invention encompasses an apparatus and method for a formula and programs not to exceed 40 minutes in length.

Another preferred embodiment of the present invention encompasses an apparatus and method for a formula with programs to specifically omit 55-65 Hz.

Another preferred embodiment of the present invention encompasses an apparatus and method for producing and employing square waves in an intermittent “fuzzy” manner. Square waves are more stimulating and are easier to control than other waveforms.

Another preferred embodiment of the present invention encompasses the use and method in which Crossover programs promote: 1) Stimulation of the overall production of endorphin. 2) Increases in brainwave amplitude and improvements in brainwave irregularities and coherence. 3) Increases and strengthening of synaptic linkages through the synchronistic firing of activated neural cells. 4) More preferably, re-patterning of neural pathways to allow for new thought formations, disruption of a wide variety of “set” responses, i.e., softening and erasure of the “stimulus and response” mechanism (“fight and flight” response), changes in cognitive function, emotion, and thought processes.

Another preferred embodiment of the present invention encompasses the use a method in which Inhibitory programs promote: 1) Stimulation of the overall production of serotonin. 2) Increases in brainwave amplitude and improvements in brainwave irregularities and coherence. 3) Increases and strengthening of synaptic linkages through the synchronistic firing of activated neural cells. 4) More preferably, treats anxiety and attentional disorders, and improves cognitive function.

Another preferred embodiment of the present invention encompasses the use a method in which Excitatory programs promote: 1) Stimulation of the overall production of dopamine. 2) Increases in brainwave amplitude and improvements in brainwave irregularities and coherence. 3) Increases and strengthening of synaptic linkages through the synchronistic firing of activated neural cells. 4) More preferably, treats depression and improves cognitive functions.

Exclusion Criteria: This device is not intended for use for anyone prone to any form of seizures, epilepsy or visual photosensitivity, or who is using a pacemaker or suffering cardiac arrhythmia or other heart disorders. The apparatus and methods are not recommended for anyone who is currently taking stimulants, tranquilizers, or psychotropic medications without the assistance of a licensed practitioner. This apparatus should not be used while under the influence of alcohol or drugs.

The apparatus and method thus have broad applicability for a wide variety of uses. This invention is particularly useful to the improvement of “cognitive functions” which encompasses but is not limited to, the ability to reason and solve problems, long and short-term memory processes, visual discrimination and perceptual skill, hand eye-coordination, visual-spatial abilities, and learning for example. Additionally, this method and apparatus is not limited to medical diseases or disorders, and may include a neurodegenerative disease, such as but not limited to Alzheimer's Disease, Parkinson's Disease, and senile dementia. It can affect pain, major depression, schizophrenia, stroke, childhood learning disorders such as attention-deficit/hyperactivity disorder (ADHD) and dyslexia, alcohol and drug abuse, and normal aging.

Thus, the invention utilizes a light emitting apparatus or goggle, and method in the form of software programs to supply white full spectrum light in an intermittent “fuzzy” manner up to 1200 Hz. accompanied by intermittent “fuzzy” sounds. It should be noted that the sound and light are not to be in coherence with each other.

The type of sound, tones, and pulses produced by the method will be controlled by the software. The volume will be controlled through the use of the light/sound device housing the software. The volume may be set at a constant level throughout the treatment, or, preferably, may be varied during the stimulation cycle or treatment, according to the individual patient's needs and diagnosis. As with the ocular apparatus positioned over the patient's eyes, the light intensity may be self-adjusted, and may derive their audio signal and power from an independent light/sound source.

The method in the form of computerized software will provide a method of increasing brain chemistry and treating a variety of disorders through the use of existing devices in which the method or programs can be downloaded. The formulas within the method are comprised of “sessions” for a variety of treatments, consisting of programs that will drive the “fuzzy” sounds and “fuzzy” light. The “fuzzy” sounds and “fuzzy” lights will not be in exact coherence with each other. The method will provide programs ranging from 1 to 1200 Hz, specifically excluding 55 Hz to 65 Hz to avoid “feedback” resulting in any form of seizure.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. An apparatus for stimulating the neurochemistry of the brain of a subject, said apparatus comprising:
a goggle member including a pair of eye shields, said eye shields having a periphery;
at least one light disposed about said periphery of each of said eye shields a distance of between 1½ and 2½ inches from the retinas of the subject; and
circuit means to drive said at least one light at a frequency of between 1-1200 Hz.

2. The apparatus for stimulating the neurochemistry of the brain of a subject of claim 1 wherein said at least one light has a varied duty cycle.

3. The apparatus for stimulating the neurochemistry of the brain of a subject of claim 2 wherein said varied duty cycle has a variance of 10% to 100% of the frequency.

4. The apparatus for stimulating the neurochemistry of the brain of a subject of claim 1 wherein said at least one light comprises between 8 and 20 lights disposed about the periphery of each of said eye shields.

5. The apparatus for stimulating the neurochemistry of the brain of a subject of claim 1 wherein said at least one light is a white light.

6. The apparatus for stimulating the neurochemistry of the brain of a subject of claim 1 wherein said at least one light is a full spectrum light.

7. The apparatus for stimulating the neurochemistry of the brain of a subject of claim 1 wherein said circuit means to drive said at least one light at a frequency of between 1-1200 Hz excludes 55-65 Hz to avoid seizure potential feedback.

8. The apparatus for stimulating the neurochemistry of the brain of a subject of claim 1 wherein said circuit means includes means for auditory stimulus.

9. The apparatus for stimulating the neurochemistry of the brain of a subject of claim 8 wherein said auditory stimulus comprises tones with a varied duty cycle.

10. The apparatus for stimulating the neurochemistry of the brain of a subject of claim 8 wherein said at least one light has a duty cycle, and said auditory stimulus varied duty cycle does not match said light duty cycle.

11. A method for stimulating the neurochemistry of the brain of a subject, said method comprising the steps of:
providing a goggle member including a pair of eye shields with at least one light disposed about the periphery of each of the eye shields a distance of between 1½ and 2½ inches from the retinas of the subject; and
driving said at least one light at a frequency of between 1-1200 Hz to stimulate the brain of the subject.

12. The method for stimulating the neurochemistry of the brain of a subject of claim 11 further including the step of varying the duty cycle of the at least one light.

13. The method for stimulating the neurochemistry of the brain of a subject of claim 11 further including the step of providing between 8 and 20 lights disposed about the periphery of each of said eye shields.

14. The method for stimulating the neurochemistry of the brain of a subject of claim 11 wherein said step of driving said at least one light at a frequency of between 1-1200 Hz to stimulate the brain of the subject further includes the step of excluding 55-65 Hz to avoid seizure potential feedback.

15. The method for stimulating the neurochemistry of the brain of a subject of claim 11 further including the step of providing an auditory stimulus to the subject.

16. The method for stimulating the neurochemistry of the brain of a subject of claim 15 wherein said step of providing an auditory stimulus comprises providing tones with a varied duty cycle.

17. The method for stimulating the neurochemistry of the brain of a subject of claim 15 wherein said step of providing an auditory stimulus comprises providing tones with a duty cycle different than the duty cycle of the light.
